(19) United States
(12) Patent Application Publication
(10) Pub. No.: US 2012/0116519 A1
(43) Pub. Date: May 10, 2012

(54) Surgical Extractor and Method of Removing Intervertebral Disc Prosthesis

(76) Inventor: Franz Copf, JR., Stuttgart (DE)

(21) Appl. No.: 13/349,818

(22) Filed: Jan. 13, 2012

Related U.S. Application Data
(63) Continuation of application No. 12/300,018, filed on Nov. 7, 2008, now abandoned.

(30) Foreign Application Priority Data
May 27, 2006 (DE) 10 2006 024 809.0

(51) Int. Cl. A61F 2/44

(52) U.S. Cl. 623/17.16

(57) ABSTRACT

A method of removing an intervertebral disc prosthesis from an intervertebral disc space includes the steps of: a) providing a surgical extractor with two legs; b) individually inserting the legs, while the legs are completely detached from each other, into the intervertebral disc space; c) rotating the legs so that they take up a locking position in which a pulling force can be applied by the legs to the intervertebral disc prosthesis; d) connecting the legs to an extraction element; and e) pulling at the extraction element so that the legs apply a pulling force to the intervertebral disc prosthesis.

### RELATED APPLICATION DATA

This patent is a continuation of co-pending U.S. application Ser. No. 12/300,018 filed Nov. 7, 2008, which is a U.S. national phase application based on international application no. PCT/EP2007/004224, filed on May 12, 2007, which claimed priority to German patent application 10 2006 024 809.0 filed on May 27, 2006. Priority benefit of these earlier filed applications is hereby claimed, and the full disclosures of these earlier filed applications are hereby incorporated by reference herein.

### BACKGROUND

1. Field of the Disclosure

The invention relates to a surgical extractor for surgical removal of intervertebral disc prostheses, which comprise two endplates with in each case one joint part.

2. Description of Related Art

Intervertebral disc prostheses are known from use which comprise two sub-elements with in each case one endplate. Each endplate has on its outward-facing side a bearing face with which the endplate is supported against a vertebra adjoining the intervertebral disc space. On the inside of each endplate there is disposed a joint part, which in the case of the one endplate takes the form of a dome-shaped joint socket and in the case of the other endplate takes the form of a corresponding dome-shaped joint head. When inserted, joint socket and joint head together form a ball-and-socket joint, such that the two endplates may be tilted in any spatial direction relative to one another.

Since such ball-and-socket joints are exposed to heavy wear due to the forces acting on them, it may be necessary to renew the entire intervertebral disc prosthesis. Replacement may also be necessary if the adjoining vertebrae become pathologically deformed, such that the intervertebral disc prosthesis is no longer optimally conformed to the physiological conditions.

The object of the invention is to provide a surgical extractor for surgical removal of intervertebral disc prostheses of the above-mentioned type.

### SUMMARY

This object is achieved by a surgical extractor which comprises two legs connected together in the manner of a fork, which may be introduced into an interspace between the two endplates of an intervertebral disc prosthesis to be extracted and which each bear at least one locking element at their free ends, which locking element, in a first position, allows introduction into the interspace between the two endplates of the intervertebral disc prosthesis and, in a second, locked position, projects vertically beyond the interspace. The two legs are connected to a common extraction means for transmitting a tensile force to the legs in the longitudinal direction thereof.

For the purpose of extraction, the legs are introduced through the interspace between the endplates of an intervertebral disc prosthesis and then locked in place. The intervertebral disc prosthesis may then be driven out of the intervertebral disc space using the extraction means.

If the legs are connected detachably to the extraction means, the legs may be introduced separately on in each case one side of the ball-and-socket joint of the intervertebral disc prosthesis and only connected to one another and to the common extraction means for the purpose of extraction once in the introduced state. This simplifies introduction of the legs.

The locking elements may be constructed in such a way that they are wider in a first transverse direction than in a second transverse direction. Thus, the two legs may in each case be introduced in one orientation, in which the locking element fits in its narrower transverse direction through the interspace between the endplates, then turned 90° into the locked position and connected together.

Preferably, the legs have a substantially round profile over their introducible length and the locking elements are in each case constructed as rigid extensions formed in T-shaped manner on the ends of the legs.

The locking elements should conveniently be so shaped that no force component tending to spread the endplates apart can be transmitted by said locking elements in the locked position, which would make it more difficult to drive the intervertebral disc prosthesis out of the intervertebral disc space.

The extraction means may advantageously comprise a shank with a limit stop and a percussive weight movable along the shank as far as the limit stop. The intervertebral disc prosthesis may then be carefully driven out in small steps in that the percussive weight is moved by the surgeon jerkily against the limit stop. The surgeon thus has good control of the force impulses which he is exerting on the intervertebral disc prosthesis.

In another advantageous exemplary embodiment, the legs have different lengths. This makes it possible to extract the intervertebral disc prosthesis from the intervertebral disc space by way of lateral access.

### BRIEF DESCRIPTION OF THE DRAWINGS

Further features and advantages of the invention are revealed by the following description of an exemplary embodiment made with reference to the drawings, in which:

FIG. 1 is a side view of an intervertebral disc prosthesis inserted between two vertebrae;

FIG. 2 is a plan view of an extractor according to the invention with an endplate of the intervertebral disc prosthesis disposed therebelow;

FIG. 3 is a side view of an individual leg of the extractor of FIG. 2;

FIG. 4 shows the intervertebral disc prosthesis of FIG. 1 with an extractor introduced from the hidden side; and

FIG. 5 is an exploded representation of the extractor shown in FIGS. 2 to 4;

FIG. 6 is a plan view based on FIG. 2 of an extractor according to another exemplary embodiment of the invention.

### DETAILED DESCRIPTION OF THE DISCLOSURE

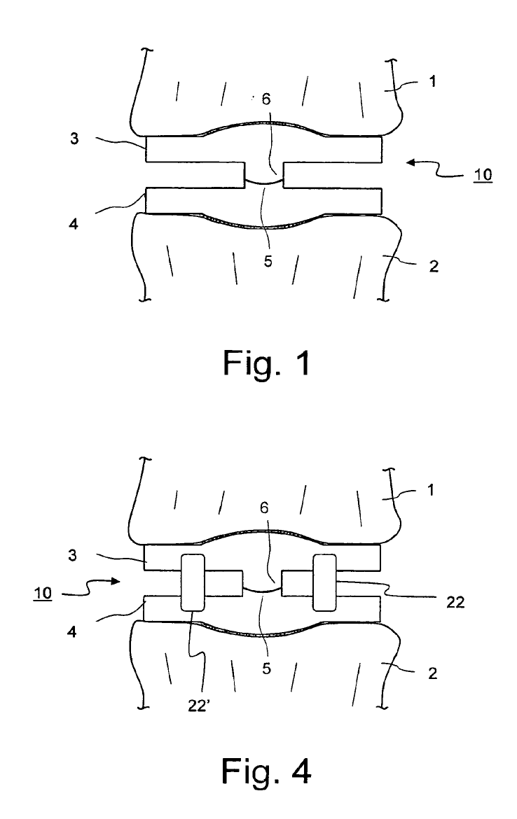

FIG. 1 shows an intervertebral disc prosthesis 10, which has been inserted into an intervertebral disc space between an upper vertebra 1 and a lower vertebra 2. The intervertebral disc prosthesis comprises an upper endplate 3 and a lower endplate 4.

On the inside of each endplate 3, 4 there is disposed a protrusion with a joint part, which in the case of the lower endplate takes the form of a dome-shaped joint socket 5 and in the case of the upper endplate 3 takes the form of a corresponding dome-shaped joint head 6. Joint socket 5 and joint head 6 together form a ball-and-socket joint, such that the two endplates 3, 4 of the intervertebral disc prosthesis 10 may be tilted in any spatial direction relative to one another.

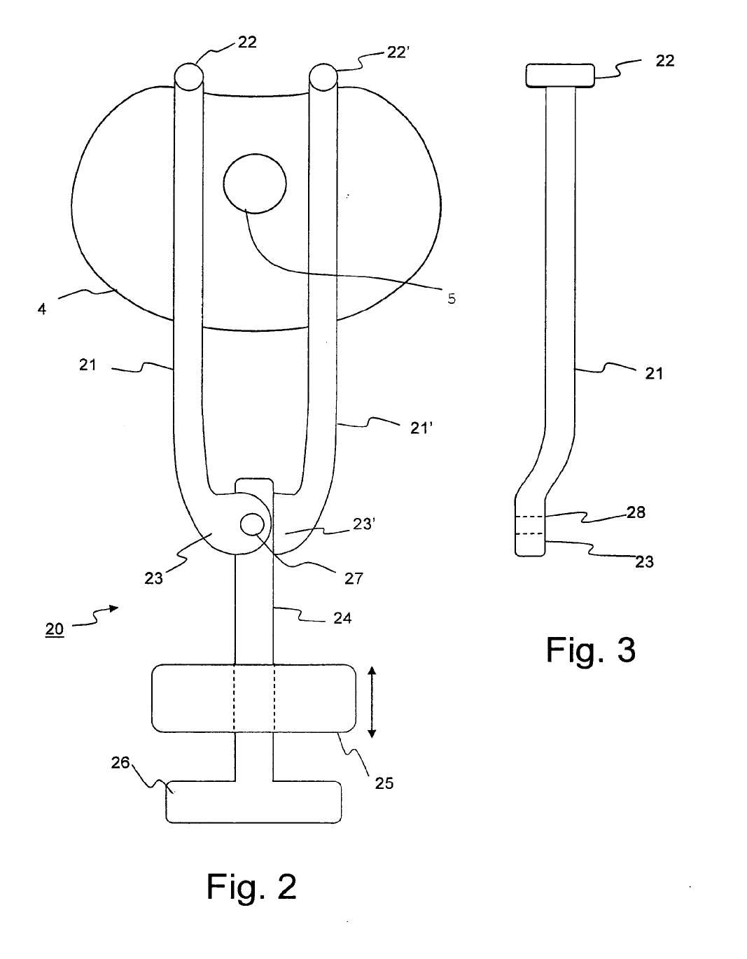

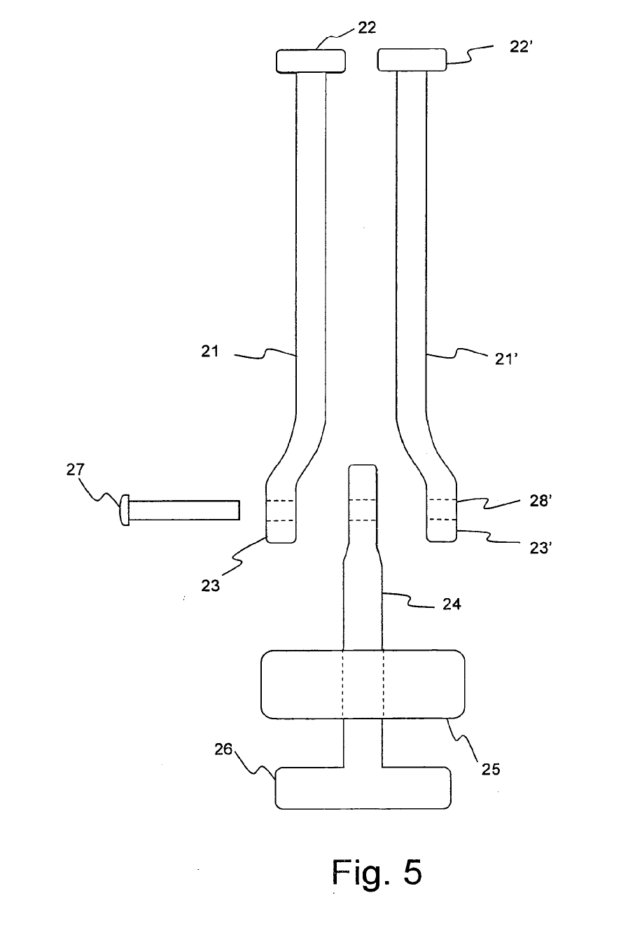

If such an intervertebral disc prosthesis 10 needs to be removed surgically for replacement, a surgical instrument known as an extractor is required. An extractor 20 according to the invention is shown schematically in plan view above the lower endplate 4 of the intervertebral disc prosthesis 10 in FIG. 2 and in exploded representation in FIG. 5.

The extractor 20 has two legs 21, 21′ of identical construction, which in each case bear at one end a rigidly formed-on, T-shaped extension 22, 22′ and at their other end in each case a laterally offset fastening lug 23, 23′. In FIG. 3 the leg 21 is shown from the side. The leg 21 and the T-shaped extension 22 have round profiles. A hole 28 in the fastening lug 23 for accommodating a pin 27 is indicated by broken lines. The T-shaped extension 22 is mounted orthogonally relative to the fastening lug 23, such that it is perpendicular to the plane generated by the legs 21, 21′ when the extractor 20 is in the assembled state.

The two legs 22, 22′ may be connected detachably by means of the pin 27 to a common shank 24, on which a movable percussive weight 25 is guided, which may be moved against a limit stop 26. It goes without saying that a screw, a pin or a similar fastening means may be selected instead of a pin 27.

When connected together the two legs 22, 22′ form a fork, which grips around the ball-and-socket joint of the intervertebral disc prosthesis 10 formed by the joint parts 5, 6. As a result of the curved portions between the fastening lugs 23, 23′ and the legs 21, 21′, the latter are arranged in one plane when they are connected together by means of the pin 27.

The dismantleable nature of the extractor 20 allows introduction of the legs 21, 21′ into the interspace between the endplates 3, 4 of an intervertebral disc prosthesis 7 to be removed. In addition, all the parts of the extractor 20 may in this way be individually cleaned and sterilised.

Preferably, the extractor 20 consists of surgical stainless steel (316L). Alternatively, other, sufficiently inert, break-resistant materials such as for example titanium or titanium alloys may of course be used.

To remove an intervertebral disc prosthesis 10, the two legs 21, 21′ are inserted individually into the gap-like interspace between the two endplates 3, 4 preferably via a ventral or lateral access canal. To this end, the legs 21, 21′ are turned in such a way that the T-shaped extension 22, 22′ is positioned in the gap direction. Once the two legs 21, 21′ have been introduced so far into the joint interspace of the intervertebral disc prosthesis 10 that the T-shaped extensions 22, 22′ project into the spinal canal on the opposite side, the legs 21, 21′ are turned by 90°, fitted together with the shank 23 and connected together by means of the pin 27.

The two extensions 22, 22′ are now positioned, as shown in FIG. 4, perpendicularly to the gap direction of the interspace between the endplates 3, 4 and grip around these from the back. The surgeon then begins to drive the intervertebral disc prosthesis 10 out of the intervertebral disc space by striking with the percussive weight 25 against the limit stop 26.

The T-shaped extensions 22, 22′ are designed such that they rest substantially flat against the end faces of the two endplates 3, 4, such that no orthogonally acting force component tending to spread the endplates 3, 4 apart is transmitted.

Alternatively, the end faces of the endplates 3, 4 may also be slightly bevelled towards the outsides thereof. If the extensions 22, 22′ are then configured in such a way that they in each case form an angle with the legs 21, 21′ which is somewhat smaller than 90°, upon driving out of the intervertebral disc prosthesis 10 a force component is transmitted which pushes the endplates 3, 4 together on the side facing the spinal canal and spreads them apart on the opposing, ventral side. A slightly wedge-shaped arrangement of the endplates thus arises, such that the intervertebral disc prosthesis 10 can slip more readily out of the intervertebral disc space.

It should be pointed out that the extractor illustrated is merely a non-limiting exemplary embodiment. An informed reader will obviously know that numerous modifications and additions to the above-described extractor are possible.

For instance, instead of a percussive mechanism any other type of extraction means may be used which is suitable for transmitting a tensile force in the longitudinal direction of the legs 21, 21′. For example, a lateral projection may be provided on the shank 24 instead of a limit stop 26 for a percussive weight 25, by means of which the surgeon may drive out the intervertebral disc prosthesis 10 using a hammer. Alternatively, a screw mechanism may also be provided, which rests against the adjoining vertebrae, in order to push the intervertebral disc prosthesis 10 out of the intervertebral disc space by screwing in one or more extraction spindles against the vertebrae.

As an alternative to the T-shaped extensions 22, 22′ with which the legs 21, 22′ are locked in place, it is also possible, for example, to provide erectable barbs or locking members mounted rotatably at the ends of the legs, which may be actuated by means of a suitable mechanism at the front end of the legs after introduction of the legs. It is also not essential for the legs 21, 21′ to be inserted over the entire width of the intervertebral disc prosthesis 10 as far as into the spinal canal. Alternatively, for example, recesses may be provided in the endplates, into which suitable locking members may engage at the ends of the extractor legs.

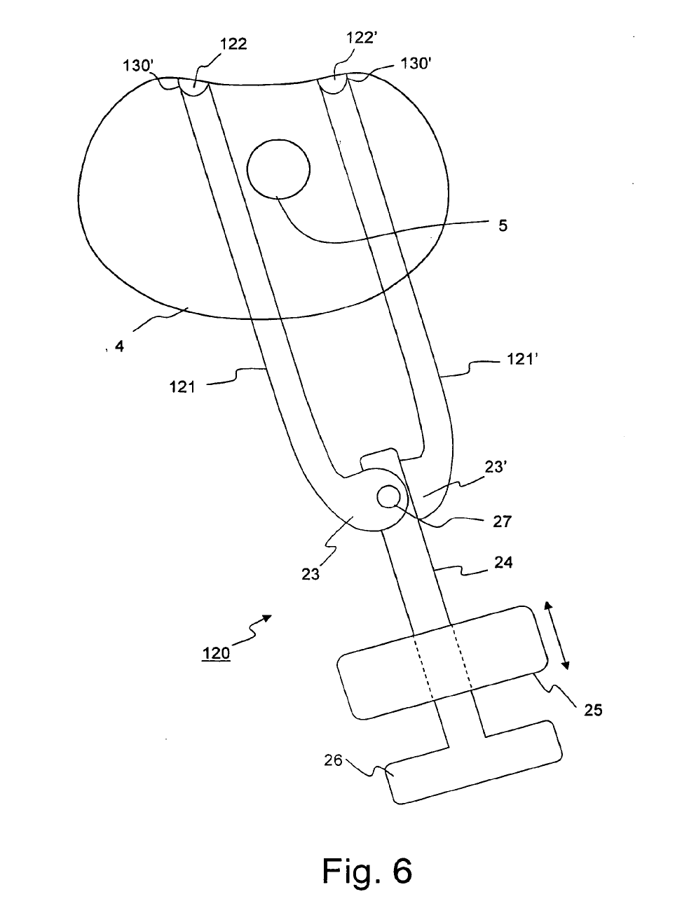

FIG. 6 is a plan view based on FIG. 2 of an extractor according to another exemplary embodiment of the invention and designated overall as 20. The extractor 120 differs from the above-described extractor 20 solely in that the legs 121, 121′ are of different lengths. In addition, the outwardly directed short legs of the T-shaped extensions 122, 122′ have a semicircular profile and the endplates 3, 4 have at their dorsal ends semicircular recesses 130, 130′, which are of complementary construction to the profile of the extensions 122, 122′.

The legs 122, 122′ of different lengths and the form-fitting engagement of the extensions 122, 122′ in the recesses 130, 130′ make it possible to pull the endplates 3, 4 out of the intervertebral disc space by way of laterally acting forces without the extensions 122, 122′ slipping against the endplates 3, 4. The semicircular shape additionally ensures that the extensions 122, 122′ do not project beyond the dorsal end of the endplates 3, 4 and in this way injure the adjoining sensitive tissue during the extraction process

Although certain surgical extractors and features thereof have been described herein in accordance with the teachings of the present disclosure, the scope of coverage of this patent is not limited thereto. On the contrary, this patent covers all embodiments of the teachings of the disclosure that fairly fall within the scope of permissible equivalents.

What is claimed is:

1. A method of removing an intervertebral disc prosthesis from an intervertebral disc space, said method comprising the following steps:
a) providing a surgical extractor that comprises two legs;
b) individually inserting the legs, while the legs are completely detached from each other, into the intervertebral disc space;
c) rotating the legs so that they take up a locking position in which a pulling force can be applied by the legs to the intervertebral disc prosthesis;
d) connecting the legs to an extraction element; and
e) pulling at the extraction element so that the legs apply a pulling force to the intervertebral disc prosthesis.

2. The method of claim 1, wherein each leg bears at a free end of it a locking element.

3. The method of claim 2, wherein the locking element is wider in a first transverse direction, which is orthogonal to a longitudinal direction of the respective leg, than in a second transverse direction which is orthogonal both to the first transverse direction and the longitudinal direction.

4. The method of claim 2, wherein the legs are inserted during step b) into an interspace that is formed between endplates of the intervertebral disc prosthesis.

5. The method of claim 4, wherein the locking element projects vertically beyond the interspace after the legs have been rotated during step c).

6. The method of claim 4, wherein the locking elements are shaped so that no force component tending to spread apart the endplates of the intervertebral disc prosthesis can be applied by said locking elements after step c).

7. The method of claim 2, wherein the locking element is a T-shaped extension formed at the free end of the respective leg.

8. The method of claim 1, wherein the legs have different lengths.

9. The method of claim 1, wherein the legs are connected to the extraction element during step d) by inserting a pin through holes that are provided in the legs and the extraction element.

10. The method of claim 1, wherein the legs are rotated by approximately 90° during step c).

11. The method of claim 1, wherein during step e) a percussive weight guided on the extraction element is moved against a limit stop that is provided on the extraction element.
